(19) United States
(12) Patent Application Publication
(10) Pub. No.: US 2024/0226483 A9
(43) Pub. Date: Jul. 11, 2024

(54) APPARATUS AND RELATED SYSTEM AND METHOD FOR CONTAINMENT AND EXHAUST OF EXHALATION

(71) Applicant: NORIG LLC, Miami, FL (US)

(72) Inventor: MAUREEN LEWARS, Miami, FL (US)

(73) Assignee: NORIG LLC, Miami, FL (US)

(21) Appl. No.: 18/277,171

(22) PCT Filed: Feb. 16, 2022
(86) PCT No.: PCT/US22/16500

§ 371 Date: Aug. 14, 2023

Related U.S. Application Data
(60) Provisional application No. 63/149,750, filed on Feb. 16, 2021.

(51) Int. Cl. A61M 16/06; A61M 11/06; A61M 16/20

(52) U.S. Cl. CPC A61M 16/0616; A61M 11/06; A61M 16/0683; A61M 16/208

(57) ABSTRACT

An apparatus adapted for use in medical respiratory treatments utilizing a nebulizer mask. The apparatus includes an outer shell configured to fit over a nebulizer mask and collect the exhalant from the nebulizer into a suction device to prevent contamination of the surrounding environment.

### CROSS-REFERENCE TO RELATED APPLICATIONS

This application is submitted under 35 U.S.C. 371 as a U.S. National Stage Entry of PCT/US22/16500, having an international filing date of Feb. 16, 2022 and is currently pending, which is a non-provisional of, and claims priority to, U.S. Patent Application Ser. No. 63/149,750, filed on Feb. 16, 2021 and now expired, which are all incorporated by reference as if fully set forth herein.

### FIELD OF THE INVENTION

The present invention generally relates to masks and, more specifically, to an apparatus, system, and related method for the containment and exhaust of contaminated air and suspended particles expelled during the exhalation cycle of a patient undergoing an aerosolized or nebulizer treatment.

### BACKGROUND OF THE INVENTION

Delivery of medication suspended in an aerosolized or nebulized saline solution is known in the art and a preferred method of administering the medication directly into the lungs of a patient. In these aerosol or nebulizer treatments, a patient is fitted with a mask that covers the nose and mouth. A tube delivers air or oxygen into a container with the saline and medicine, creating an aerosolized mist, suspending the medicine and saline as droplets in the mist directed into the mask. Once in the mask, the patient need only breathe normally to inhale the medicine directly into the lungs.

A necessary by-product of known nebulizer treatment systems is that the masks also have vent holes or other similar features in order to allow excess aerosol to be vented out from the mask. Unfortunately, this also allows for any exhalation to also escape from the mask and into the surrounding environment. If the patient is harboring any airborne virus, bacteria, or other pathogen, then that pathogen is also expelled into the surrounding environment and can directly infect others that inhale that air, or even just remain latent on nearby surfaces.

It would be desirable to have an apparatus, system, and related method for the containment and exhaust of contaminated air and suspended particles expelled during the exhalation cycle of a patient undergoing a nebulizer treatment. Especially in the current era of highly infectious airborne pathogens, such as SARS-CoV-2, there currently exists a need in the industry for an apparatus and related system and method for the containment and exhaust of contaminated air and suspended particles expelled during the exhalation cycle of a patient undergoing a nebulizer treatment.

### SUMMARY OF THE INVENTION

The present invention is a specialized mask that, when fitted over a patient's face, covers the nose and mouth, delivers the nebulized medication, and safely exhausts the patient's exhaled air without any, or at least minimal, contamination of the surrounding environment. The core components of the invention are a mask, one or more straps, an air/oxygen intake port, a nebulizer device, and an exhaust port, which are generally configured as follows:

the mask is a molded, medical-grade plastic that is shaped to cover a patient's nose and mouth; one or more straps, preferably elastic, are threaded through the mask and are configured to secure the mask on the patient's head; a nebulizer device is located near the bottom of the mask (near the patient's chin) and has an inlet, an outlet, and a reservoir to hold the saline-plus-medication solution; the air/oxygen intake port is configured to sealably receive a tube delivering the air/oxygen into the nebulizer device; the inlet of the nebulizer device is sealably communicative with the air/oxygen intake port of the mask and the outlet of the nebulizer device is disposed to expel the nebulized solution into the inside of the mask (the side of the mask that creates a sealed space in front of the patient's nose and mouth); the exhaust port is disposed on the mask and is communicative with the inside of the mask and the outside of the mask, where the portion of the exhaust port that is outside of the mask is configured to sealably attach a suction tube for removal of the exhaled air.

The invention can be used to deliver aerosol/nebulizer treatments to patients where the inhalation and exhalation occur in a closed cycle such that contaminated exhaled air is not transmitted into the surrounding environment or to healthcare workers in close proximity.

It should be noted that the present invention provides a significant safety improvement over the current state of the art. Using known art nebulizer treatment masks, a nurse or respiratory therapist would be almost immediately exposed to contaminated air as soon as the patient was fitted with the standard nebulizer mask. Even if the healthcare worker wore personal protective equipment (PPE), the contaminated particles would likely adhere to the PPE and risk being transported to others. Nebulizer treatments are often required many times around the clock and thus create many exposure risks. The present invention avoids this altogether since the exhaled air is automatically suctioned out of the room and not expelled into the surrounding environment.

Furthermore, it is an object of the present invention that multiple embodiments are presented and are still within the scope of the invention. For example, in addition to the preferred embodiment disclosed above, it is contemplated that the invention may be modified to include a retrofit version. Such an embodiment would be configured to fit over an existing nebulizer mask, with the exhaust port configured to suction the exhaled air that escapes from the nebulizer mask's vent holes. Yet other embodiments include versions where the location and arrangement of the air/oxygen inlet and the exhaust port are disposed in different positions on the mask in order to perform more efficiently, or to provide a more comfortable or ergonomic positioning of the ports and their connected tubing.

Other features that are considered as characteristic for the invention are set forth in the drawings and preferred embodiment.

Although the invention is illustrated and described herein as embodied in an apparatus for the containment and exhaust of contaminated air and suspended particles expelled during the exhalation cycle of a patient undergoing a nebulizer treatment, the invention should not be limited to the details shown in those embodiments because various modifications and structural changes may be made without departing from the spirit and scope of the invention. Furthermore, while the invention is described within the context of the disclosed apparatus and related system and method, one of ordinary skill in the art will realize that the invention may be sized and configured as appropriate for other applications such as for pediatric use, adult use, or even animal use.

The construction and method of operation of the invention and additional objects and advantages of the invention is best understood from the following description of specific embodiments when read in connection with the accompanying drawings.

### BRIEF DESCRIPTION OF THE DRAWINGS

In the accompanying figures, like reference numerals refer to identical or functionally similar elements throughout the separate views. The accompanying figures, together with the detailed description below, are incorporated in and form part of the specification and serve to further illustrate various embodiments and to explain various principles and advantages all in accordance with the present invention, in which:

FIG. 1 is a front view of an embodiment of the invention.

FIG. 2 is a front view of another embodiment of the invention.

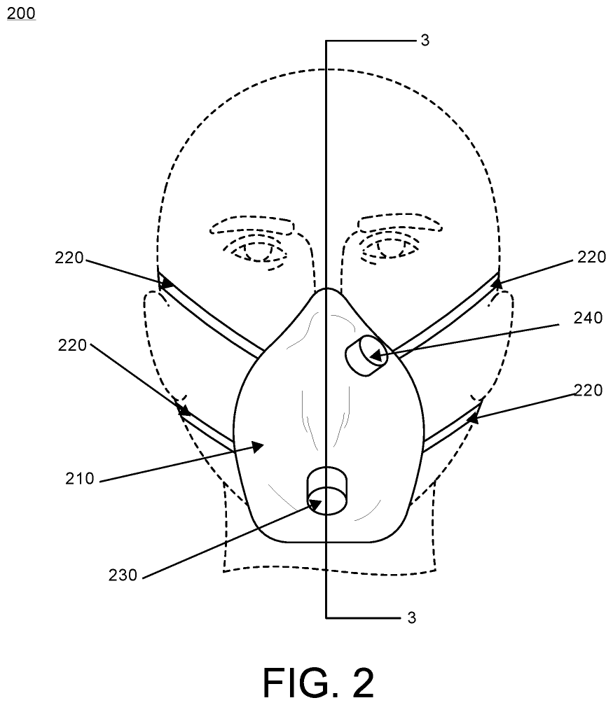

FIG. 3 is a cross sectional view of FIG. 2.

FIG. 4 shows a front view of another embodiment of the invention.

While the invention as claimed can be modified into alternative forms, specific embodiments thereof are shown by way of example in the drawings and will herein be described in detail. It should be understood, however, that the drawings and detailed description thereto are not intended to limit the invention to the particular form disclosed, but on the contrary, the intention is to cover all modifications, equivalents and alternatives falling within the scope of the present invention.

### DETAILED DESCRIPTION

In this specification and in the appended claims and drawings, words and phrases have the meanings commonly attributed to them in the relevant art except as otherwise specified herein. The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, “air” means any gas that is introduced into the apparatus, inhaled by the wearer, and subsequently exhaled and exhausted from the apparatus. It is contemplated that “air” could be, by example and not limitation, room air, filtered or purified air, oxygen, or an air/oxygen mixture.

As used herein, the terms “aerosol,” “nebulizer,” and their grammatical equivalents are used interchangeably.

The present invention may address one or more of the problems and deficiencies of the known art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein. While certain aspects of conventional technologies and methods in the relevant art have been discussed to facilitate disclosure of the invention, Applicant in no way disclaims these technical aspects or methods, and it is contemplated that the claimed invention may encompass one or more of the conventional technical aspects or methods discussed herein.

Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. Specifically, component names, types, and values, as depicted in the exemplary schematic diagrams, are not intended to limit the scope of the present invention and are presented only as possible embodiments.

Preferably, embodiments of the present invention include, at least, a structure configured to create a sealed volume when worn by a user and an exhaust port adapted to be connected to a suction device such that, when worn by a user, the sealed volume collects and contains the user's exhalant and said exhalant is suctioned from the sealed volume through the exhaust port.

Other embodiments further include a nebulizer mask adapted to cover a nose and mouth of the user and defining a nebulizer volume, where the nebulizer volume is entirely contained within the sealed volume of the structure, and said nebulizer mask is further adapted to receive an aerosol into the nebulizer volume and expel said exhalant from the user into the sealed volume.

The vacuum setting of a connected suction device would have to be adjusted by medical professionals to make certain that the suction does not remove the nebulized treatment within the nebulized before the patient is able to breathe in the medication. The suction, for example, may be set to “low” (10-80 mm Hg) and preferably in the range of 20-30 mm Hg. In this way, the aerosol may adequately collect within the nebulizer volume for inhalation by the user, while the exhalant that collects in the sealed volume is suctioned safely away.

The following exemplary embodiments are disclosed, however, it is to be understood that the invention may be carried out in multiple embodiments, and features from one embodiment may be used in another embodiment, and still remain within the scope of the present invention.

### Embodiment 1

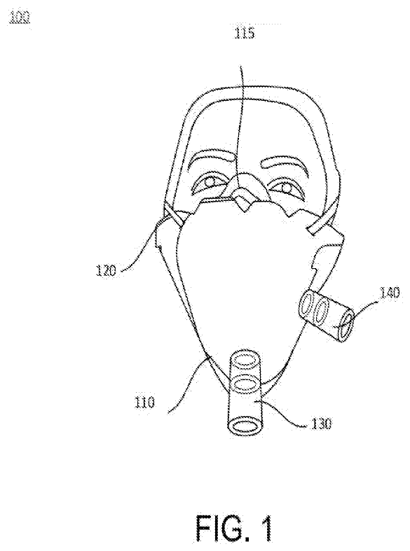

An embodiment of the present invention, as shown in FIG. 1, is an apparatus 100 for the containment and exhaust of contaminated air and suspended particles expelled during the exhalation cycle of a patient undergoing a nebulizer treatment. FIG. 1 shows the apparatus 100 that is worn on the face (shown for reference) of a user. Furthermore, FIG. 1 shows that the apparatus 100 is worn over a standard nebulizer mask 115 (shown for reference) as is currently known in the art. A standard nebulizer mask, as is known in the art, is worn over a user's nose and mouth and defines a nebulizer volume. The standard nebulizer mask has an inlet to receive the aerosol or nebulizer treatment into the nebulizer volume, and also includes one or more vent holes, typically near the user's nose, to allow the user's exhaled breath (exhalant) to be expelled from the nebulizer volume.

The apparatus 100 is made up of the following components: an outer shell 110 configured to fit over a standard nebulizer mask 115, at least one elastic strap 120, an inhalation tube 130, and an exhaust tube 140. The outer shell 110 is adapted to cover the standard nebulizer mask and, more importantly, the one or more vent holes. In this way, the outer shell 110 creates and defines a sealed volume that collects and contains the exhalant from the user to be suctioned out via the exhaust tube. In this embodiment, as shown in FIG. 1, the inhalation tube 130 is adapted to be sealably communicative with a corresponding inlet in the standard nebulizer mask 115.

### Embodiment 2

In this second embodiment, the apparatus 200 combines the features and components of the previous embodiment and also adds the features of a nebulizer mask component. In this embodiment, the patient does not need to wear two masks. Rather, the apparatus 200 includes all of the components necessary to both administer a nebulizer treatment and connect to a vacuum system for the safe excretion of the exhaled air.

The core components of this embodiment of the invention are a mask 210, one or more straps 220, an inlet port 230, and an exhaust port 240. The mask 210 is a molded, medical-grade plastic that is shaped create two compartments, or volumes: a nebulizer volume 340 configured to cover a patient's nose and mouth, and a sealed volume 330 that is configured to contain the nebulizer volume 340. Additionally, the nebulizer volume 340 is sealably communicative with the inlet port 230 and further contains one or more exhaust holes 335 that are communicative with the sealed volume 330.

One or more straps 220, preferably elastic, are threaded through the mask 210 and are configured to secure the mask 210 on the patient's head.

The inlet port 230 is adapted to sealably receive a nebulizer device. As is known in the art, a standard nebulizer device (not shown for compactness of disclosure) has an inlet, an outlet, and a reservoir to hold the saline-plus-medication solution. Air/oxygen is introduced into the nebulizer device and when the inlet of the nebulizer device is sealably communicative with the inlet port 230, the nebulized solution is expelled into the nebulizer volume 340 (that side of the mask that creates a sealed space in front of the patient's nose and mouth).

The exhaust port 240 is disposed on the mask 210, and is communicative between the sealed volume 330 (the inside of the mask) and the outside of the mask 210, where the portion of the exhaust port 240 that is outside of the mask 210 is adapted to sealably attach a suction tube (not shown) for removal of the exhalant that has been collected and contained in the sealed volume 330. The exhaust port is preferably located at the top of the mask (near the patient's nose) for efficient suction of the exhalant without compromising the delivery of medication. However, various locations and orientations for the exhaust port may be manufactured as per market needs, and still be within the scope of the present invention.

### Embodiment 3

In another embodiment of the present invention, the mask 110 is similar to the apparatus described in Embodiment 1; however, in this third embodiment, the exhaust port 140 is located near the bottom of the mask 110 to allow easy alignment of the hose such that it does not present additional pressure to the patient's face by the imbalance of the weight of the overall device 100 when it is fastened to the patient's face via the elastic straps 120.

### Embodiment 4

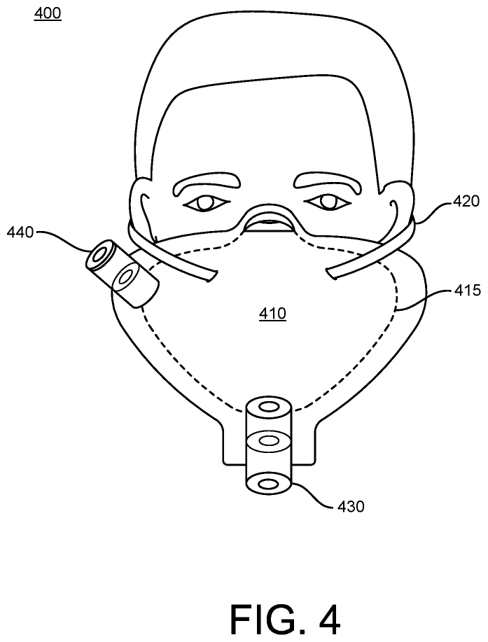

Referring to FIG. 4, there is shown an apparatus 400 for the containment and exhaust of contaminated air and suspended particles expelled during the exhalation cycle of a patient undergoing a nebulizer treatment. As shown in FIG. 4, the apparatus 400 includes, at least, an outer shell (mask) 410 configured to fit over a standard nebulizer mask 415 (shown in dashed lines underneath the outer shell 410), at least one strap 420 to secure the apparatus in place, an inhalation tube 430 for inhaling the aerosol mist for treatment, and at least one exhaust tube 440 for expelling the exhaust. The exhaust mechanism, whether it is a tube 440 or ventilation hole, is disposed on the inside layer of the outer shell 410.

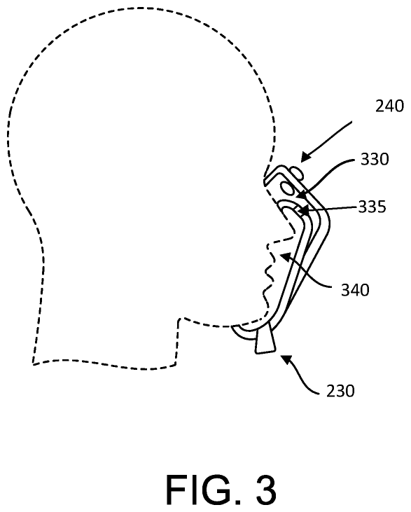

Similar to the embodiment shown in FIG. 1, the outer shell 410 is sized to fit over a regular mask and nebulizing device 415 such as those commonly used in hospitals. The wider clamshell-like shape allows plenty of room for a negative pressure cavity (gap) such as the sealed volume 330 shown in FIG. 3, over and surrounding the inhalation tube of the inner mask 415. This gap allows the nebulized medicine (aerosol) to collect.

Just as in the embodiment shown in FIG. 1, the exhaust port 440 (exhaust tube) is configured to accept a hose or tube that is connected to a suction device. FIG. 4 shows an embodiment where the exhaust port 440 is disposed on the upper right-hand side of the mask 410 where it can be easily connected to a suction canister or other vacuum system. The placement of the exhaust port 440 can be changed to accommodate the positioning of suction canisters. In this manner the exhaust expelled through the exhaust port 440 never gets into the atmosphere, assuring the safety of the patient, as well as the safety of those around the patient.

The embodiment shown in FIG. 4 features a square bottom portion 411 over the inhalation tube 430, allowing an easier means of attaching the inhalation tube 430 to a nebulizer or oxygen tank or canister. The extra room in the square configuration also accommodates an adapter (not shown) so that the apparatus 400 can be used with multiple inhalation treatment components.

Additional Optional Components and Features:

Any of the embodiments of the present invention may also have one or more of each of the following (disjunctive list): one or more compliant seals or gaskets to seal between the outer shell (mask) and the patient's face; multiple elastic straps, ear loops, or other equivalent fixing means; a one-way valve in the inhalation (intake) tube (port); a one-way valve in the exhaust tube (port); internal ducting to separate intake air flow from exhaust airflow.

Related System:

It should further be noted that a related system includes the previously described apparatus (including any of the non-limiting, exemplary embodiments) in concert with tubing to connect the mask to an air or oxygen supply, as well as, tubing to connect the mask to a suction device. In embodiments, the system may also include seals, gaskets, or other means of controlling the airflow through the mask.

Related Method:

A method related to the use of the apparatus for the containment and exhaust of contaminated air and suspended particles expelled during the exhalation cycle of patient undergoing a nebulizer treatment comprises the steps of: providing the previously described apparatus (including any of the non-limiting, exemplary embodiments); placing the apparatus on a patient covering the nose and mouth; providing an air supply; providing a suction; attaching the air supply to the inhalation tube; attaching the suction to the exhaust tube; introducing an airflow via the air supply; and creating a suction via the suction. In this way, the apparatus of the present invention is utilized for the containment and exhaust of contaminated air and suspended particles expelled during the exhalation cycle of a patient undergoing a nebulizer treatment.

The description of the present disclosure has been presented for purposes of illustration and description, but is not intended to be exhaustive or limited to the invention in the form disclosed. Many modifications and variations will be apparent to those of ordinary skill in the art without departing from the scope and spirit of the disclosure. In particular, features or components from one embodiment can be used with another embodiment. The embodiments were chosen and described to best explain the principles of the disclosure and the practical application, and to enable others of ordinary skill in the art to understand the invention for various embodiments with various modifications as are suited to the particular use contemplated.

What is claimed is:

1. An apparatus comprising:
a structure configured to create a sealed volume when worn by a user; and
an exhaust port disposed on the structure and sealably communicative through the structure with the sealed volume and further adapted to be connected to a suction device,
whereby, when worn by a user, the sealed volume collects and contains an exhalant from the user and said exhalant is suctioned from the sealed volume through the exhaust port.

2. The apparatus of claim 1 further comprising a nebulizer mask adapted to cover a nose and mouth of the user and defining a nebulizer volume, where the nebulizer volume is entirely contained within the sealed volume of the structure, and said nebulizer mask is further adapted to receive an aerosol into the nebulizer volume and expel the exhalant from the user into the sealed volume.

3. An apparatus adapted for use in medical respiratory treatments that utilize a nebulizer mask, the apparatus comprising:
an outer shell adaptable to enclose a nebulizer mask and configured to collect an exhalant from the nebulizer mask;
an inhalation tube disposed through the outer shell and configured to be sealably communicative with an inlet of the nebulizer mask;
an exhaust tube disposed through the outer shell and configured to be communicative with the exhalant from the nebulizer mask and adapted to sealably connect to a suction device; and
at least one elastic strap attached to the outer shell and configured to keep the outer shell in place over the nebulizer mask.
